SEQUENCE LISTING (6 sequences)

SEQ ID NO: 1 - protein, 33 aa, Artificial Sequence
  synthetic p53 immunogenic fragment, immunogenic epitope
GSRAHSSHLKSKKGQSTSRHKKLMFKTEGPDSD

SEQ ID NO: 2 - protein, 20 aa, Artificial Sequence
  synthetic p53 immunogenic fragment, immunogenic epitope
MDDLMLSPDDIEQWFTEDPG

SEQ ID NO: 3 - protein, 20 aa, Artificial Sequence
  synthetic IGF2BP2 immunogenic fragment, immunogenic epitope
QFENYSFKISYIPDEEVSSP

SEQ ID NO: 4 - protein, 472 aa, Homo sapiens
  selenium binding protein 1, isoform 1 (SBP1, SELENBP1), 56 kDa selenium binding protein (SBP56, SP56), LPSB
MATKCGNCGPGYSTPLEAMKGPREEIVYLPCIYRNTGTEAPDYLATVDVDPKSPQYCQVIHRLPMPNLKDELHHSGWNTCSSCFGDSTKSRTKLVLPSLISSRIYVVDVGSEPRAPKLHKVIEPKDIHAKCELAFLHTSHCLASGEVMISSLGDVKGNGKGGFVLLDGETFEVKGTWERPGGAAPLGYDFWYQPRHNVMISTEWAAPNVLRDGFNPADVEAGLYGSHLYVWDWQRHEIVQTLSLKDGLIPLEIRFLHNPDAAQGFVGCALSSTIQRFYKNEGGTWSVEKVIQVPPKKVKGWLLPEMPGLITDILLSLDDRFLYFSNWLHGDLRQYDISDPQRPRLTGQLFLGGSIVKGGPVQVLEDEELKSQPEPLVVKGKRVAGGPQMIQLSLDGKRLYITTSLYSAWDKQFYPDLIREGSVMLQVDVDTVKGGLKLNPNFLVDFGKEPLGPALAHELRYPGGDCSSDIWI

SEQ ID NO: 5 - protein, 393 aa, Homo sapiens [sequence omitted]

SEQ ID NO: 6 - protein, 599 aa, Homo sapiens
  insulin-like growth factor 2 mRNA-binding protein-2 (IGF2BP-2), hepatocellular carcinoma autoantigen p62, VICKZ family member 2 (VICKZ2), IMP-2, IMP2
MMNKLYIGNLSPAVTADDLRQLFGDRKLPLAGQVLLKSGYAFVDYPDQNWAIRAIETLSGKVELHGKIMEVDYSVSKKLRSRKIQIRNIPPHLQWEVLDGLLAQYGTVENVEQVNTDTETAVVNVTYATREEAKIAMEKLSGHQFENYSFKISYIPDEEVSSPSPPQRAQRGDHSSREQGHAPGGTSQARQIDFPLRILVPTQFVGAIIGKEGLTIKNITKQTQSRVDIHRKENSGAAEKPVTIHATPEGTSEACRMILEIMQKEADETKLAEEIPLKILAHNGLVGRLIGKEGRNLKKIEHETGTKITISSLQDLSIYNPERTITVKGTVEACASAEIEIMKKLREAFENDMLAVNQQANLIPGLNLSALGIFSTGLSVLSPPAGPRGAPPAAPYHPFTTHSGYFSSLYPHHQFGPFPHHHSYPEQEIVNLFIPTQAVGAIIGKKGAHIKQLARFAGASIKIAPAEGPDVSERMVIITGPPEAQFKAQGRIFGKLKEENFFNPKEEVKLEAHIRVPSSTAGRVIGKGGKTVNELQNLTSAEVIVPRDQTPDENEEVIVRIIGHFFASQTAQRKIREIVQQVKQQEQKYPQGVASQRSK

(19) United States
(12) Patent Application Publication
(10) Pub. No.: US 2013/0196350 A1
(43) Pub. Date: Aug. 1, 2013

(54) SENSITIVITY AND SPECIFICITY FOR OVARIAN CANCER

(71) Applicant: Bio-Rad Laboratories, Inc., Hercules, CA (US)

(72) Inventors: Steven R. Binder, Berkeley, CA (US); Michelle Delanoy, Danville, CA (US); Audrey Arjomandi, San Francisco, CA (US); John Flanagan, Walnut Creek, CA (US)

(73) Assignee: Bio-Rad Laboratories, Inc., Hercules, CA (US)

(21) Appl. No.: 13/755,234

(22) Filed: Jan. 31, 2013

Related U.S. Application Data
(60) Provisional application No. 61/593,084, filed on Jan. 31, 2012.

(51) Int. Cl. G01N 33/68; G01N 33/74

(52) U.S. Cl. CPC G01N 33/6893; G01N 33/6854; G01N 33/74
USPC 435/7.23

(57) ABSTRACT

Improved ovarian cancer assays are provided.

### CROSS-REFERENCE TO RELATED PATENT APPLICATIONS

The present patent application claims benefit of priority to U.S. Provisional Patent Application No. 61/593,084, filed Jan. 31, 2012, which is incorporated by reference.

### BACKGROUND OF THE INVENTION

Cancer Antigen 125 (CA-125) is the only marker that has been FDA cleared for ovarian cancer (OVCA), and it is used clinically for monitoring treatment response. While CA-125 may be ordered “off-label” when there is suspicion of ovarian cancer (“screening”), the low prevalence of this disease, combined with the false positive rate, means that the positive predictive value (PPV) of an abnormal result is quite low (around 1%).

### BRIEF SUMMARY OF THE INVENTION

Methods of detecting the presence or absence of cancer (e.g., ovarian cancer) in an individual human are provided. In some embodiments, the methods comprise, detecting the level of the following agents in a biological sample from the individual:

a. CA-125; and
b. insulin-like growth factor binding protein 2 (IGFBP-2) and/or prolactin and/or osteopontin; and
correlating the level of the agents to the presence, absence, or stage of ovarian cancer in the individual wherein the correlating comprises using the IGFBP-2 or prolactin or osteopontin levels as a confirmatory criterion for higher than normal levels of CA-125.

In some embodiments, the methods further comprise detecting in a biological sample from the individual the level of at least one autoantibody specific for a target antigen protein, wherein an elevated level of the autoantibody specific for the target antigen protein is indicative of cancer.

In some embodiments, the correlating comprises determining whether the level of CA-125 is below about 30 IU/mL serum, between about 35 and 100 IU/mL serum, or over about 100 IU/mL serum, and whether IGFBP-2 or prolactin or osteopontin levels are above normal levels, wherein the presence of ovarian cancer is indicated by:

a CA-125 level over about 100 IU/mL; or
a CA-125 level between about 30 and 100 IU/mL and an IGFBP-2 and/or prolactin and/or osteopontin level are above the normal level.

In some embodiments, the anti-CA-125 antibody and the IGFBP-2 and/or prolactin and/or osteopontin antibody are linked to the same solid support. In some embodiments, the solid support is a bead.

In some embodiments, the anti-CA-125 antibody and the IGFBP-2 and/or prolactin and/or osteopontin antibody are linked to different solid supports. In some embodiments, the solid support is a plurality of beads, the beads comprising a bead linked to the anti-CA-125 antibody and a bead linked to the anti-IGFBP-2 and/or prolactin and/or osteopontin antibody, wherein the bead linked to the anti-CA-125 antibody is distinguishable from the bead linked to the anti-IGFBP-2 and/or prolactin and/or osteopontin antibody by flow cytometry.

In some embodiments, the at least one autoantibody is detected by capturing the autoantibody on a solid support and detecting specific binding of the autoantibody to the autoantibody's respective target antigen protein or immunogenic fragment thereof. In some embodiments, the autoantibody is captured by the target antigen protein, or an immunogenic fragment thereof, linked to the solid support, and the specific binding of the autoantibody to the target antigen protein is detected by detecting binding of an anti-human IgG antibody to the autoantibody. In some embodiments, the autoantibodies for the target antigen protein are separately captured by:

the target antigen protein; and

the immunogenic fragment thereof; and

the detecting comprises separately detecting binding of the autoantibodies to the target antigen protein and to the immunogenic fragment thereof.

In some embodiments, more than one target antigen protein for more than one different autoantibodies are linked to the solid support, thereby detecting the level of more than one autoantibody in the sample.

In some embodiments, the solid support is a bead.

In some embodiments, the autoantibody target antigen protein is SBP1, p53, and/or insulin-like growth factor 2 mRNA-binding protein 2 (IGF2BP2).

In some embodiments, the immunogenic fragment comprises SEQ ID NO:1, 2, or 3.

Kits for detecting cancer in a human individual are also provided. In some embodiments, the kit comprises anti-CA-125 antibody; and an anti-IGFBP-2 and/or anti-prolactin and/or anti-osteopontin antibody. In some embodiments, the antibody(ies) is linked to a solid support. In some embodiments, the solid support is a bead.

In some embodiments, the kit further comprises: a target antigen protein, or an immunogenic fragment thereof, that specifically detects an autoantibody that occurs at a higher rate in individuals having cancer compared to individuals not having cancer. In some embodiments, the kit comprises the antigen and the immunogenic fragment thereof. In some embodiments, the antigen is SBP1, p53, and/or IGF2BP2. In some embodiments, the immunogenic fragment comprises SEQ ID NO:1, 2, or 3. In some embodiments, the antigen is linked to a solid support. In some embodiments, the solid support is a bead.

In some embodiments, the kit further comprises one, two, or more different antigens and/or immunogenic fragments thereof, each of which specifically detect a different autoantibody that occurs at a higher rate in individuals having cancer compared to individuals not having cancer. In some embodiments, the two or more different antigens, or immunogenic fragments thereof, are linked to the same solid support. In some embodiments, the solid support is a bead.

In some embodiments, the anti-CA-125 antibody and the anti-IGFBP-2 and/or anti-prolactin and/or anti-osteopontin antibody are linked to the same solid support. In some embodiments, the solid support is a bead. In some embodiments, the anti-CA-125 antibody and the anti-IGFBP-2 and/or anti-prolactin and/or anti-osteopontin antibody are linked to different solid supports. In some embodiments, the solid support is a plurality of beads, the beads comprising a bead linked to the anti-CA-125 antibody and a bead linked to the IGFBP-2 antibody and/or anti-prolactin and/or anti-osteopontin antibody, wherein the bead linked to the anti-CA-125 antibody is distinguishable from the bead linked to the IGFBP-2 antibody or anti-prolactin or anti-osteopontin antibody by flow cytometry.

In some embodiments, the kit further comprises an anti-human IgG antibody. In some embodiments, the anti-human IgG antibody is linked to a detectable label.

Methods of detecting cancer in an individual are also provided. In some embodiments, the methods comprise,

detecting the level of cancer-associated autoantibodies in a sample derived from an individual,

wherein the autoantibodies bind to a target antigen protein selected from SBP1, p53, and/or IGF2BP2, wherein the detecting comprises:

capturing the autoantibodies with the target antigen protein and determining the quantity of autoantibodies captured by the target antigen protein; and

capturing the autoantibodies with an immunogenic fragment of the target antigen protein and determining the quantity of autoantibodies captured by the immunogenic fragment,

wherein the individual has cancer if the quantity of autoantibodies captured by the target antigen protein and the quantity of autoantibodies captured by the immunogenic fragment is above a normal level.

In some embodiments, the antigen is selected from SBP1, p53, and/or IGF2BP-2. In some embodiments, the antigen or immunogenic fragment thereof is linked to a solid support. In some embodiments, the solid support is a bead.

In some embodiments, the immunogenic fragment comprises SEQ ID NO:1, 2, or 3.

In some embodiments, the target antigen protein; and the immunogenic fragment; are linked to different solid supports.

In some embodiments, the autoantibodies bind to the antigen and the bound autoantibodies are quantified by contacting the bound autoantibodies with an anti-human IgG antibody.

In some embodiments, the cancer is ovarian cancer.

Kits for detecting cancer in a human individual are also provided. In some embodiments, the kit comprises, target antigen protein, wherein the antigen is selected from SBP1, p53, and/or IGF2BP2, and an immunogenic fragment of the target antigen protein.

In some embodiments, the immunogenic fragment comprises SEQ ID NO:1, 2, or 3.

In some embodiments, the antigen and immunogenic fragment thereof is linked to a solid support. In some embodiments, the solid support is a bead.

In some embodiments, the antigen, and the immunogenic fragment are linked to different solid supports.

In some embodiments, the antigen or immunogenic fragment thereof and the antibody are in the same tube or vessel.

In some embodiments, the antigen or immunogenic fragment thereof and the antibody are in different tubes or vessels.

In some embodiments, the kit further comprises an anti-human IgG antibody. In some embodiments, the anti-human IgG antibody is linked to a detectable label.

In some embodiments, the kit further comprises a capture agent specific for CA-125.

Methods of detecting the presence or absence of cancer (e.g. ovarian cancer) in an individual human are also provided. In some embodiments, the method comprises, detecting the level of the following agents in a biological sample from the individual:

a. CA-125; and
b. two or more of: autoantibodies specific for SBP1, autoantibodies specific for p53, and/or autoantibodies specific for IGF2BP2;
correlating the level of the agents to the presence, absence, or stage of ovarian cancer in the individual.

In some embodiments, detecting the autoantibodies comprises contacting a sample to an antigen selected from SBP1, and/or IGF2BP2, and/or p53 and/or a polypeptide comprising an immunogenic fragment thereof, and contacting the sample to one or more immunogenic fragment of the antigen.

Methods of detecting the presence or absence of p53 autoantibodies in a sample from human blood are also provided. In some embodiments, the method comprises contacting the sample to a polypeptide comprising SEQ ID NO:2 or 1; and detecting the quantity of binding of antibodies from the sample to SEQ ID NO:2 or 1, thereby detecting presence or absence of p53 autoantibodies in the sample. In some embodiments, the method further comprises contacting the sample to a full-length p53 polypeptide, and detecting the quantity of binding of antibodies from the sample to the full-length p53 polypeptide.

### DETAILED DESCRIPTION OF THE INVENTION

### I. Introduction

Markers are provided, which when used in combination, provide for improved specificity and sensitivity in ovarian cancer detection. In one embodiment, one or more biomarker is used as a confirmatory marker to a second marker (e.g., CA-125) to detect ovarian cancer. Examples of confirmatory biomarkers include, e.g., insulin-like growth factor binding protein 2 (IGFBP-2), prolactin, or osteopontin. As described in more detail below, it has been surprisingly discovered that detection of elevated levels of IGFBP-2 in a human can act as a confirmatory test when elevated levels of the marker CA-125 are found.

Further, it has been discovered that detection of autoantibodies for several antigens is useful to increase specificity of CA-125 assays by detecting individuals who are negative for CA-125 but positive for the one or more autoantibodies. For example, it has been surprisingly discovered that the presence of autoantibodies for selenium binding protein 1 (SBP1) or p53, can be indicative of ovarian cancer even when CA-125 levels are not elevated. Thus, the combination of the detection of one or more of these autoantibodies with the detection of CA-125 (and optionally a confirmatory biomarker) allows for improved ovarian cancer detection specificity compared to detection of CA-125 alone.

In addition, it has been surprisingly found that false positive detection of ovarian cancer based on the presence of autoantibodies can be reduced by detecting the autoantibodies with a specific immunopeptide from the antigenic protein for the autoantibodies in combination with the entire antigenic protein. Elevated levels of autoantibodies that bind to the immunogenic fragment of the antigen acts as a confirmation for elevated levels of autoantibodies bound to the full-length antigen.

II. Confirmatory Biomarkers Improve Specificity of Detection of Ovarian Cancer with CA-125

As noted above, it has been discovered that use of confirmatory markers in combination with monitoring of CA-125, can improve the specificity of CA-125 detection. Studies of CA-125 have reported that at a value of 100 U/mL, the CA-125 specificity is 99.9%, meaning one false positive result per 1000 results in a healthy population (see, e.g., Bon, G. G., et al., Am. J. Obstet. Gynecol. 174:107-14 (1996); Skates, S J, et al., J. Clin. Oncol. 21:206s-210s (2003)). Further, CA-125 has a specificity of at most 98% at the commonly used cut off of 35 IU/mL. This means that for every 100 healthy results essentially 2 out of 100 will be >35 but only 1/1000 will be over 100. Given low disease prevalence, and the fact that most ovarian cancer patients have CA-125>100 when diagnosed, many of the patients with serum values between 35-100 IU/mL will turn out to be false positives.

It has been discovered that Insulin-Like Growth Factor Binding Protein-2 (IGFBP-2), prolactin or osteopontin in combination with CA-125 improves the specificity of cancer detection, especially for patients who have somewhat elevated CA-125 levels. While IGFBP-2, prolactin and osteopontin are not elevated in all ovarian cancer patients, the presence of any of these proteins can be used to differentiate false positive elevation of CA-125 due to other clinical conditions from true positive elevation due to ovarian cancer.

As noted above, while extremely high CA-125 levels (e.g., over 100 IU/ml) are generally indicative of the presence of cancer, somewhat elevated levels (e.g., 35-100 IU/ml) do not always indicate the presence of cancer. IGFBP-2 measurement is of particular use in differentiating cancer when patients have these somewhat elevated levels (e.g., percentile levels at about 98-99.9 of normal values) of CA-125. Similarly prolactin or osteopontin measurement is also useful when patients have somewhat elevated CA-125 levels. Thus, in some embodiments, detection of the presence or absence of cancer in an individual comprises detection of at least CA-125 and an additional protein marker (e.g., IGFBP-2 and/or prolactin and/or osteopontin), wherein either of the following indicate the presence of cancer:

CA-125 levels over 100 IU/ml regardless of levels of other protein biomarkers; or

CA-125 levels between about 30 or 35 and 100 IU/ml and levels of an additional marker (e.g., IGFBP-2 or prolactin and/or osteopontin) are above normal levels.

“Above normal” marker levels refer to levels of the marker (e.g., IGFBP-2, prolactin and/or osteopontin) that are above the 99th percentile observed in normal healthy people. Because these marker are used as a confirmatory criterion (i.e., to confirm results observed for CA-125), the cutoff for these three marker can be different and less stringent from what would be used if the same markers were used as a screening criterion.

While the above description is provided with reference to CA-125, it should be appreciated that other biomarkers besides CA-125 (e.g., including but not limited to mesothelin and CA 15-3) can be managed to maximize their utility by accepting unconditionally a value above the 99.9^{th} percentile as a positive result and accepting values between the 98th percentile and the 99.9^{th} percentile as positive only if at least one marker selected from IGFBP-2 or prolactin or osteopontin is above normal levels.

Detection of CA-125

CA-125 can be detected in any format known in the art. CA-125, also known as “Mucin 16” or “Muc16” in the art, is a glycoprotein. See, e.g., Jacobs, Human Reproduction 4(1):1-12 (1989). Detection of CA-125 refers to detection of the intact CA-125 protein, or fragments thereof that are indicative of the presence of the intact CA-125 protein. A number of formats for detection of CA-125 can be used according to the invention. Generally, a capture agent, immobilized on a solid support, is used to capture CA-125 from the sample. The capture agent can be, for example, an antibody. Alternatively, the capture agent is a non-antibody protein. A large number of scaffolds for generating non-antibody proteins with high binding specificities are known or can be generated. See, e.g., Bestes, et al., Proc Natl Acad Sci USA. 96(5): 1898-1903 (1999); U.S. Pat. No. 7,115,396; U.S. Pat. No. 7,018,801; and US Patent Publication No. 2005/0221384. Once captured from a sample, CA-125 can be detected using a detection agent. The detection agent can be, for example, an antibody or non-antibody protein that specifically binds CA-125. The detection agent can be directly labeled (e.g., with a fluorescent or other label) or can be detected indirectly, e.g., via a secondary antibody that is detectably labeled, or by enzymatic reaction in embodiments where an enzyme (e.g., HRP) is linked to the detection or secondary antibody, or via affinity linkers such as biotin/streptavidin to link the detection reagent to the label.

In some embodiments, CA-125 in a sample is initially captured by contacting the sample with the capture agent immobilized on a solid support under conditions to allow for binding of CA-125, if present in the sample, to the immobilized capture agent. The presence of the captured CA-125 is then detected, optionally following one or more wash step to remove non-binding components of the sample.

In some embodiments, the solid support is a bead or particle (used interchangeably herein). Exemplary beads include but are not limited to those that can be sorted by flow cytometry, e.g., Luminex beads. Once CA-125 in the sample is captured, the particles are recovered and separated from some or all of the remaining reagents in the mixture. For example, in some embodiments, the sample is removed from the particles by washing the particles in an appropriately buffered solution. Particles can be recovered by any method known in the art. In some cases, the particles are pelleted by centrifugation and the remaining sample (i.e., the supernatant) is removed from the particles. In some embodiments, the particles are responsive to a magnetic field and a magnetic field is applied such that the liquid in a sample is removed while the particles adhere to a reaction vessel wall, separating the remaining liquid from the particles. The particles can optionally be washed, e.g., one or more times with an appropriate buffer, if desired.

The captured CA-125 is subsequently detected and quantified. In some embodiments, the CA-125 can be detected by incubating the captured CA-125 with a labeled antibody or non-antibody protein that specifically binds to CA-125, thereby allowing the labeled antibody to bind to the captured CA-125. Excess labeled antibody can be subsequently removed, and the remaining labeled antibody (associated with the particles) is detected and optionally quantified. The presence and quantity of the label can be used to estimate the amount of CA-125 in the original sample, for example, by comparing the quantity of label to a calibration curve based on known amounts of CA-125, as is well known in the art.

Alternative methods for detecting CA-125 can also be used. Without intending to limit the invention to a particular method of detecting CA-125, one alternative is a competition assay. In these embodiments, CA-125 immobilized on a solid support (e.g., a particle) is incubated with a sample as well as an exogenous CA-125 that is optionally labeled, thus allowing for competition of the exogenous CA-125 with any endogenous CA-125 in the sample. Reduction in signal from the label associated with the exogenous CA-125 is thus related to increased amount of endogenous CA-125 in the sample.

Detection of Other Biomarkers

Biomarkers described herein can be detected in any desired format. Insulin-Like Growth Factor Binding Protein-2 (IGFBP-2) is encoded by the human IGFBP2 gene and is described in, e.g., Roghani M, et al. Growth Regul. 1(3): 125-30 (1993); Ho P J, and Baxter R C Clin. Endocrinol. (Oxf) 46(3): 333-42 (1997). Prolactin is a peptide found in human milk. A representative prolactin protein sequence can be found as NP_{—}000939.1 in NCBI. Osteopontin is also known as uropontin, nephropontin, SPP1/CALPHA1 fusion, urinary stone protein, early T-lymphocyte activation 1, osteopontin/immunoglobulin alpha 1 heavy chain constant region fusion protein, and secreted phosphoprotein 1 (osteopontin, bone sialoprotein I, early T-lymphocyte activation 1) and is a human gene product. A representative osteopontin protein sequence can be found as NP_{—}000573.1 in NCBI.

Detection of a confirmatory marker can include detection of the intact marker protein, or fragments thereof that are indicative of the presence of the intact protein. A number of formats for detection of marker proteins can be used and formats as described with regard to CA-125 above can also be applied for detection of the confirmatory marker protein. For example, in some embodiments, the capture agent used to capture the marker protein from the sample can be linked to the same or a different solid support as bound to the CA-125 solid support. In embodiments in which different solid supports are used, the solid supports linked to the CA-125 capture agent can be distinguished from solid supports linked to the marker protein capture agent by a physical characteristic of the solid support.

III. Autoantibodies Improve Sensitivity for Detection of Ovarian Cancer with CA-125

While CA-125 is elevated in many patients with ovarian cancer, some tumors do not express CA-125 and thus are not detected by an assay based on CA-125 detection alone. In addition, patients with stage 1 and stage 2 disease are less likely to have an elevated level of CA-125, making early stage detection more difficult when CA-125 is used alone. Finally, CA-125 is less frequently elevated in some types of ovarian cancer. It has been surprisingly discovered that detection of autoantibodies specific for one or more antigen (SBP1, p53, or insulin-like growth factor 2 mRNA-binding protein 2 (IGF2BP2)) are useful for detecting the types and stages of cancer that are sometimes missed when CA-125 detection alone is employed. Thus, detection of these autoantibodies is useful in combination with CA-125 for detection of cancer, including ovarian cancer. As shown in Table 1, detection of autoantibodies specific for SBP1, p53, or IGF2BP2 has been shown to detect the presence of cancer in ovarian cancer patients who have a CA-125 level less than 100 IU/ml. Detection of SBP1 autoantibodies (Barua, A. et al., Amer. J. Reproduct. Immunol. 57:243-249 (2007); WO 2011/035101), p53 autoantibodies (Anderson, K., et al., Cancer Epidemiol Biomarkers Prey 19:859-868 (2010)), and IGF2BP2 (Zhang J Y et al, Clin Immunol. 2001 August; 100(2):149-56) have been described before.

The full-length sequences for these (SBP1, p53 and IGF2BP2) antigens are known.

| | |
|---|---|
| Selenium- | MATKCGNCGPGYSTPLEAMKGPREEIVYLPCIYRNTGTEAPDYLATVDVDPKSPQYCQVIHR |
| binding | LPMPNLKD |
| protein 1 | ELHHSGWNTCSSCFGDSTKSRTKLVLPSLISSRIYVVDVGSEPRAPKLHKVIEPKDIHAKCE |
| (SBP1)-SEQ | LAFLHTSH |
| ID NO: 4 | CLASGEVMISSLGDVKGNGKGGFVLLDGETFEVKGTWERPGGAAPLGYDFWYQPRHNVMIST |
| | EWAAPNVL |
| | RDGFNPADVEAGLYGSHLYVWDWQRHEIVQTLSLKDGLIPLEIRFLHNPDAAQGFVGCALSS |
| | TIQRFYKN |
| | EGGTWSVEKVIQVPPKKVKGWLLPEMPGLITDILLSLDDRFLYESNWLHGDLRQYDISDPQR |
| | PRLTGQLF |
| | LGGSIVKGGPVQVLEDEELKSQPEPLVVKGKRVAGGPQMIQLSLDGKRLYITTSLYSAWDKQ |
| | FYPDLIRE |
| | GSVMLQVDVDTVKGGLKLNPNELVDEGKEPLGPALAHELRYPGGDCSSDIWI |
| p53 isoform a | MEEPQSDPSVEPPLSQETFSDLWKLLPENNVLSPLPSQAMDDLMLSPDDIEQWFTEDPGPDE |
| SEQ ID NO: 5 | APRMPEAA |
| | PPVAPAPAAPTPAAPAPAPSWPLSSSVPSQKTYQGSYGFRLGFLHSGTAKSVTCTYSPALNK |
| | MFCQLAKT |
| | CPVQLWVDSTPPPGTRVRAMAIYKQSQHMTEVVRRCPHHERCSDSDGLAPPQHLIRVEGNLR |
| | VEYLDDRN |
| | TFRHSVVVPYEPPEVGSDCTTIHYNYMCNSSCMGGMNRRPILTIITLEDSSGNLLGRNSFEV |
| | RVCACPGR |
| | DRRTEEENLRKKGEPHHELPPGSTKRALPNNTSSSPQPKKKPLDGEYFTLQIRGRERFEMFR |
| | ELNEALEL |
| | KDAQAGKEPGGSRAHSSHLKSKKGQSTSRHKKLMFKTEGPDSD |
| IGF2BP2 | MMNKLYIGNLSPAVTADDLRQLFGDRKLPLAGQVLLKSGYAFVDYPDQNWAIRAIETLSGKV |
| (accession | ELHGKIME |
| number | VDYSVSKKLRSRKIQIRNIPPHLQWEVLDGLLAQYGTVENVEQVNTDTETAVVNVTYATREE |
| NP_006539.3) | AKIAMEKL |
| SEQ ID NO: 6 | SGHQFENYSFKISYIPDEEVSSPSPPQRAQRGDHSSREQGHAPGGTSQARQIDFPLRILVPT |
| | QFVGAIIG |
| | KEGLTIKNITKQTQSRVDIHRKENSGAAEKPVTIHATPEGTSEACRMILEIMQKEADETKLA |
| | EEIPLKIL |
| | AHNGLVGRLIGKEGRNLKKIEHETGTKITISSLQDLSIYNPERTITVKGTVEACASAEIEIM |
| | KKLREAFE |
| | NDMLAVNQQANLIPGLNLSALGIFSTGLSVLSPPAGPRGAPPAAPYHPFTTHSGYFSSLYPH |
| | HQFGPFPH |
| | HHSYPEQEIVNLFIPTQAVGAIIGKKGAHIKQLARFAGASIKIAPAEGPDVSERMVIITGPP |
| | EAQFKAQGRIFGKLKEEN |
| | FFNPKEEVKLEAHIRVPSSTAGRVIGKGGKTVNELQNLTSAEVIVPRDQTPDENEEVIVRII |
| | GHFFASQTAQRKIREIVQQVKQQEQKYPQGVASQRSK |

It will be appreciated that variants (e.g., SNPs and mutations) of the proteins are known or can be readily developed and can therefore be used in place of the originally identified full-length protein.

Exemplary immunogenic fragments include, but are not limited to, those listed below:

| SEQ ID | | |
|---|---|---|
| NO: | Antigen | Immunogenic fragment |
| 1 | p53 | GSRAHSSHLKSKKGQSTSRHKKLMFKTEGPDSD |
| 2 | p53 | MDDLMLSPDDIEQWFTEDPG |
| 3 | IGF2BP2 | QFENYSFKISYIPDEEVSSP |

A number of formats for detection of autoantibodies can be used in the methods described herein. In some embodiments, a capture agent, immobilized on a solid support, is used to capture the autoantibodies. The capture agent can be, for example, an antigen that the autoantibody specifically recognizes. The autoantibody capture agent can be the full-length capture agent or a polypeptide comprising a fragment thereof comprising an epitope recognized by the antibody to be detected. As described below in more detail, in some embodiments, a full length antigen and an immunogenic fragment of the antigen are separately used to detect the autoantibodies, where the immunogenic fragment results act to confirm results based on the full-length antigen. In some embodiments, the fragments are at least, e.g., 6, 8, 10, 12, 15, 20, 25, 30, 40, 50 or more contiguous amino acids of the full length antigen. Alternatively, the capture agent can be an antibody that binds human IgG. Once captured, the autoantibody can be detected using a labeled detection agent. The detection agent can be, for example, whichever of (1) the antigen (or immunogenic fragment) or (2) antibody that binds human IgG that was not used in the capture step.

Peptide epitopes can be identified by epitope mapping. One approach is to synthesize overlapping peptides, for example 20 residues in length, with a six residue overlap, which cover the entire primary sequence of a protein. However, depending on the position of the epitope in the sequence, it can be desirable to use different length peptide sequences to best define the minimal epitope present in a protein, and to ensure that an epitope is not missed because it was artificially split between overlapping peptides. In some embodiments, the immunogenic fragments are 20 amino acids in length or greater, for example, 21, 22, 23, 24, 25, 26, 27, 28, 29, or 30 or more amino acids in length. In some embodiments, the immunogenic fragments are in the range of from 20 amino acids to 50 amino acids in length, e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. In some embodiments, immunogenic fragments may be joined together, or modified to include additional amino acids at the N-terminus or C-terminus. In some embodiments, the sequence is extended on the N and/or C terminals to provide additional amino acid residues that are present in the flanking sequences in the protein. This can more closely mimic the primary, and to a certain extent, the secondary structure environment of the epitope. Additionally, residues including but not limited to one or more glycines or gamma amino butyric acid, can be appended to either terminus to provide a spacer to minimize steric interactions with, for example, a solid phase used in an immunoassay. Spacer length is often varied to determine empirically the best structure.

In some embodiments, autoantibodies in a sample are initially captured by contacting the sample with the capture agent immobilized on the solid support under conditions to allow for binding of the autoantibodies, if present in the sample, to the immobilized antigen. The presence of the captured autoantibodies is then detected. The capture agent can be linked directly to the solid support or can be linked indirectly via a linker. The linkage can be covalent or non-covalent (e.g., via biotin/streptavidin affinity or the like).

In some embodiments, the solid support is a bead or particle. Exemplary beads include but are not limited to beads (particles) that can be sorted by flow cytometry, including but not limited to, Luminex beads. In some embodiments where multiple different autoantibodies are detected, different antigens are linked to different beads, optionally different beads that can be sorted by flow cytometry. Once autoantibodies in the sample are captured, the particles are recovered and separated from some or all of the remaining reagents in the mixture. For example, in some embodiments, the sample is removed from the particles by washing the particles in an appropriately buffered solution. Particles can be recovered by any method known in the art. In some cases, the particles are pelleted by centrifugation and the remaining sample (i.e., the supernatant) is removed from the particles. In some embodiments, the particles are responsive to a magnetic field and a magnetic field is applied such that the liquid in a sample is removed while the particles adhere to a reaction vessel wall, separating the remaining liquid from the particles. The particles can optionally be washed, e.g., one or more times with an appropriate buffer, if desired.

The captured autoantibodies are subsequently detected and optionally quantified. In embodiments where the capture step is specific autoantibodies (and thus does not significantly capture other IgG antibodies), the autoantibodies can be detected by incubating the captured autoantibodies with a labeled antibody that specifically binds to human IgG, thereby allowing the labeled antibody to bind to the captured autoantibodies. Excess labeled antibody is subsequently removed, and the remaining labeled antibody (now associated with the particles) is detected and optionally quantified. The presence and quantity of the label can be used to estimate the amount of autoantibodies in the original sample, for example, by comparing the quantity of label to a calibration curve based on known amounts of autoantibodies as is well known in the art.

Any type of anti-human IgG antibody can be used in the assay for detection of autoantibodies. Anti-human antibodies can be generated by administering human IgG, optionally with an adjuvant, to a non-human animal thereby stimulating production of antibodies in the animal that bind to human IgG. Optionally, anti-human IgG antibodies can be generated in vitro, e.g., by screening phage display antibody libraries or other antibody libraries. The anti-human IgG antibodies can be for example, mouse, rat, rabbit, goat, donkey or other non-human animal antibodies.

As noted above, the full-length antigen, and a fragment thereof comprising an epitope recognized by an autoantibody, can be used as separate capture agents or optionally as separate detection agents. Linear epitopes are typically about six amino acids, though this can vary somewhat. In order to mimic linear epitopes present in a protein, synthetic peptides can be made corresponding to the sequence. In some embodiments, this sequence is extended on the N and/or C terminals to provide additional amino acid residues that are present in the flanking sequences in the protein. This can more closely mimic the primary, and to a certain extent, the secondary structure environment of the epitope. Additionally, residues including but not limited to one or more glycines or gamma amino butyric acid, can be appended to either terminus to provide a spacer to minimize steric interactions with, for example, a solid phase used in an immunoassay. Spacer length is often varied to determine empirically the best structure.

Because of the variable nature of the epitope and the potential effects due to the flanking sequences, in some embodiments, one can use peptides that vary in length by extending the N or C terminals by a certain number of residues. Another approach utilizes repeating peptide epitopes, or alternating epitopes with intervening spacer residues. The length of these peptides is often varied according to the number of repeating units desired.

Peptide epitopes can be identified by epitope mapping. One approach is to synthesize overlapping peptides, for example 20 residues in length, with a six residue overlap, which cover the entire primary sequence of a protein. However, depending on the position of the epitope in the sequence, it is often desirable to use different length peptide sequences to best define the minimal epitope present in a protein, and to ensure that an epitope is not missed because it was artificially split between overlapping peptides.

Peptides can vary greatly in their chemical properties, particularly in regard to hydrophobicity and ionic nature. For example, in order to modulate the properties of a highly hydrophobic epitope, neutral and hydrophilic residues can be added to one or both termini. This will result in a more hydrophilic, and thus accessible epitope for antibody binding, and a generally more soluble peptide.

In some embodiments, peptides derived from hydrophobic regions of a protein can interact strongly with the surface of a bead to which they are coupled due to hydrophobic or other interactions. Ionic interactions of charged peptides with a bead surface can also occur. This can result in the inaccessibility or diminished binding of a peptide to antibodies that would typically be able to bind to it in the context of the native protein.

To overcome the undesirable interactions of peptides with solid phase supports used in immunoassays, the peptides can be modified in several ways. One way is to substitute hydrophobic residues in the peptide with hydrophilic ones, in order to reduce or minimize the hydrophobic interactions, and increased peptide accessibility. Similarly, charged peptide residues can be substituted with noncharged residues to eliminate ionic interactions with the solid phase. Accordingly, in some embodiments, the “antigens” used in the assay are not exactly fragments of the full-length antigen sequence, but instead are highly similar fragments, i.e., having at least two sequences of at least 3 or 4 amino acids that are identical to the full length antigen, linked by one or two amino acids that correspond to a position in the full-length antigen, but is different from the amino acid at that position in the full-length antigen.

Additionally, residues in the peptide can be substituted with different residues which can improve the immunoreactivity of the peptide relative to the native structure. The amino acid residues that can be substituted, such as proline, typically result in a peptide with less freedom of movement or rotation, although, in many cases, the amino acids for substitution that provide optimal immunoreactivity must be determined empirically, or in some cases using molecular modeling. In some cases, non-natural amino acids can be substituted effectively for natural amino acids. Peptides can be modified by adding spacer groups of a variety of structures to position the peptide epitope further from the solid phase and minimize steric hindrance.

Peptides can be synthesized to reflect post translation modifications that are present in the native protein. Modifications include but are not limited to phosphorylation, glycosylation, cyclization, citrullinization, etc. to mimic the form present in the native molecule, particularly at a specific site in the protein.

Peptides can also be cyclized in several manners, such as via disulfide or amide bond formation, which provides a more rigid structure, and a more favorable binding epitope for antibodies.

IV. Improved Specificity of Autoantibody Detection by Using Immunoreactive Antigen fragments

It has been further discovered that specificity of autoantibody-based cancer detection can be improved by detecting autoantibodies that bind to a full length antigen and separately detecting autoantibodies that bind to one or more immunogenic fragment of the same antigen. Detection of the autoantibodies using two different proteins (the full length protein and a fragment thereof) has been found to improve specificity of the results. That is, using an immunopeptide from the full length antigen, in combination with the full-length antigen, reduces the rate of false positive detection compared to use of full-length antigen alone.

In these aspects, a full length antigen as well as one or more immunogenic fragment of the antigen are used such that the amount of autoantibodies binding to the full length antigen can be differentiated from the amount of autoantibodies binding a particular immunogenic fragment.

While one immunogenic fragment can be used in this aspect, in some embodiments, two or more immunogenic fragments are separately used to detect the autoantibodies, and in some embodiments, the amount of autoantibodies binding each fragment is separately detectable. For example, in some embodiments, the full length antigen is linked to a first solid support and the immunogenic fragment is linked to a second solid support such that the two solid supports can be distinguished. In some embodiments, the antigen and the immunogenic fragment are linked to separate types of beads that can be separated based on mass, fluorescence, or other characteristics, thereby allowing for separate detection of autoantibodies binding thereto.

In some embodiments, the detected autoantibodies are specific for SBP1, p53, and/or IGF2BP2.

### III. Methods of Detection

As noted above, in some embodiments, the methods comprise the combined detection of CA-125 and a marker protein and/or detection of certain autoantibodies. In some embodiments, each component to be detected is captured onto a different solid support. For example, in some embodiments, the assay involves a first solid support linked to a capture agent for CA-125, a second solid support linked to a capture agent for the confirmatory marker protein(s) (e.g., IGFBP-2, prolactin and/or osteopontin), and optionally a third (or more) solid support(s), linked to a capture agent for a first autoantibody (with additional autoantibodies, if detected, each detected by a capture agent on a different solid support). As explained above, in addition, there can be a separate immunogenic fragment of the antigen for separately capturing and detecting the autoantibodies. Alternatively, the assay can be designed such that capture agents or more than one component are linked to the same solid support. The presence, absence, or level of each component is determined by using different labels to detect the specific binding between the detection agent for each component. For example, in some embodiments, a first solid support (e.g., a bead) is linked to both a capture agent for CA-125 and a capture agent for the marker protein. This solid support is then contacted to a biological sample such that CA-125 or the marker protein binds their respective capture agents and the remaining sample is washed away. The specific, differently-labeled, detection agents are applied, thereby allowing quantitative detection of both CA-125 and the marker protein using one solid support/reaction. Similarly, where one desires to detect more than one autoantibody in a sample, multiple different antigens can be linked to one solid support, thereby allowing for detection of autoantibodies for any autoantibody that specifically binds the antigens on the solid support. The level of the auto-antigens can then be detected with one general detection agent (e.g., and anti-human IgG antibody) or alternatively, each autoantibody can be detected with a separate detection agent.

In some embodiments, the different particles can be distinguished by flow cytometry by a characteristic independent of the presence or absence of the component to be detected (e.g., independent of CA-125, confirmatory marker protein, or autoantibodies) on the respective particles. In these embodiments, the particles can be sorted and the amount of label associated with each particle can be determined, thereby allowing for simultaneous determination of the amount of different components from the sample on different particles.

One can correlate the results of the assay to the presence of ovarian cancer using cut-off values (also referred to as threshold values). Where a component of the sample is higher than a set cut-off value, the sample is “positive” for that component, which is indicative of cancer. In some embodiments, the threshold value distinguishes between one diagnosis and another. For example, a threshold value can represent the level of a component generally found to distinguish between cancer samples and normal samples with a desired level of sensitivity and specificity. Cut-offs can be, for example, those values above the 95^{th}, 98^{th}, 99^{th}, 99.9^{th} or other percentile of healthy values.

In some embodiments, the threshold value can vary depending on the assays used to measure a component. Comparisons between a level of a component in a sample and a threshold value can be performed in any way known in the art. For example, a manual comparison can be made or a computer can compare and analyze the values to correlate to the likely presence of ovarian cancer.

While particular cut-off values are set forth above, it will be understood that other cut-off values can be established depending on how the correlation is established. In some embodiments, an algorithm is used to establish cut-off values and/or to correlate the patient data to prediction of the presence or absence of ovarian cancer in the subject. Algorithmic techniques for relating biomarkers of the present disclosure include but are not limited to a linear regression technique, a nonlinear regression technique, an ANOVA technique, a neural network technique, a genetic algorithm technique, a support vector machine technique, a tree learning technique, a nonparametric statistical technique, a forward, backward, and/or forward-backward technique, and a Bayesian technique. The word “technique” is intended to encompass a process in which a predictor is built by using patient exemplar pairs of biomarkers and phenotypes, and then refining such predictor algorithm in an iterative process by testing a version of the algorithm on unseen (“test”) data and making changes to mathematical coefficients of such algorithm in such a way to increase the accuracy and specificity of the predictor algorithm.

In some embodiments, the methods comprise recording a diagnosis, prognosis, risk assessment or classification, based on the level of components determined from an individual. Any type of recordation is contemplated, including but not limited to electronic recordation, e.g., by a computer.

This invention is applicable to the analysis of sample biological fluids, including but not limited to, physiological fluids such as whole blood, plasma, serum, urine, and saliva.

### V. Detectable Labels

The labels used can be any label that is capable of directly or indirectly emitting or generating detectable signal. In some embodiments, the labels are fluorophores. As noted in more detail below, if desired, fluorophores may also be incorporated into the particles themselves to distinguish one group of particles from another. A vast array of fluorophores are reported in the literature, and many are readily available from commercial suppliers to the biotechnology industry. Literature sources for fluorophores include Cardullo et al., Proc. Natl. Acad. Sci. USA 85: 8790-8794 (1988); Dexter, D. L., J. of Chemical Physics 21: 836-850 (1953); Hochstrasser et al., Biophysical Chemistry 45: 133-141 (1992); Selvin, P., Methods in Enzymology 246: 300-334 (1995); Steinberg, I. Ann. Rev. Biochem., 40: 83-114 (1971); Stryer, L. Ann. Rev. Biochem., 47: 819-846 (1978); Wang et al., Tetrahedron Letters 31: 6493-6496 (1990); Wang et al., Anal. Chem. 67: 1197-1203 (1995).

The following is a list of examples of fluorophores:
4-acetamido-4′-isothiocyanatostilbene-2,2′ disulfonic acid
acridine
acridine isothiocyanate
5-(2′-aminoethyl)aminonaphthalene-1-sulfonic acid (EDANS)
4-amino-N-[3-vinylsulfonyl)phenyl]naphthalimide-3,5 disulfonate
N-(4-anilino-1-naphthyl)maleimide anthranilamide
BODIPY
Brilliant Yellow
coumarin
7-amino-4-methylcoumarin (AMC, Coumarin 120)
7-amino-4-trifluoromethylcoumarin (Coumaran 151)
cyanine dyes
cyanosine
4′,6-diaminidino-2-phenylindole (DAPI)
5′,5″-dibromopyrogallol-sulfonaphthalein (Bromopyrogallol Red)
7-diethylamino-3-(4′-isothiocyanatophenyl)-4-methylcoumarin diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansylchloride)
4-(4′-dimethylaminophenylazo)benzoic acid (DABCYL)
4-dimethylaminophenylazophenyl-4′-isothiocyanate (DABITC)
eosin
eosin isothiocyanate
erythrosin B
erythrosin isothiocyanate
ethidium
5-carboxyfluorescein (FAM)
5-(4,6-dichlorotriazin-2-yl)aminofluorescein (DTAF)
2′,7′-dimethoxy-4′5′-dichloro-6-carboxyfluorescein (JOE)
fluorescein
fluorescein isothiocyanate
fluorescamine
IR144
IR1446
Malachite Green isothiocyanate
4-methylumbelliferone
ortho cresolphthalein
nitrotyrosine
pararosaniline
Phenol Red
phycoerythrin (including but not limited to B and R types)
o-phthaldialdehyde
pyrene
pyrene butyrate
succinimidyl 1-pyrene butyrate
quantum dots
Reactive Red 4 (Cibacron™ Brilliant Red 3B-A)
6-carboxy-X-rhodamine (ROX)
6-carboxyrhodamine (R6G)
lissamine rhodamine B sulfonyl chloride rhodamine
rhodamine B
rhodamine 123
rhodamine X isothiocyanate
sulforhodamine B
sulforhodamine 101
sulfonyl chloride derivative of sulforhodamine 101 (Texas Red)
N,N,N′,N′-tetramethyl-6-carboxyrhodamine (TAMRA)
tetramethyl rhodamine
tetramethyl rhodamine isothiocyanate (TRITC)
riboflavin
rosolic acid
lanthanide chelate derivatives

If desired, the fluorophores (or other labels) can be used in combination, with a distinct label for each analyte. In some embodiments, however, a single label is used for all labeled binding members, the assays being differentiated solely by the differentiation parameter distinguishing the individual particle groups from each other.

The attachment of any of these fluorophores to the binding members described above to form assay reagents for use in the practice of this invention is achieved by conventional covalent bonding, using appropriate functional groups on the fluorophores and on the binding members. The recognition of such groups and the reactions to form the linkages will be readily apparent to those skilled in the art.

Methods of, and instrumentation for, flow cytometry are known in the art, and can be used in the practice of the present invention. Flow cytometry in general resides in the passage of a suspension of particles (or cells) in as a stream through a light beam and coupled to electro-optical sensors, in such a manner that only one particle at a time passes the region of the sensors. As each particle passes this region, the light beam is perturbed by the presence of the particle, and the resulting scattered and fluoresced light are detected. The optical signals are used by the instrumentation to identify the subgroup to which each particle belongs, along with the presence and amount of label, so that individual assay results are achieved. Descriptions of instrumentation and methods for flow cytometry are found in the literature. Examples are McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence Immunoassays Using Flow Cytometry Instrumentation,” Clinical Flow Cytometry, Bauer, K. D., et al., eds. (Baltimore, Md., USA: Williams and Williams, 1993), pp. 535-544; Lindmo et al., “Immunometric Assay Using Mixtures of Two Particle Types of Different Affinity,” J. Immunol. Meth. 126: 183-189 (1990); McHugh, “Flow Cytometry and the Application of Microsphere-Based Fluorescence Immunoassays,” Immunochemica 5: 116 (1991); Horan et al., “Fluid Phase Particle Fluorescence Analysis: Rheumatoid Factor Specificity Evaluated by Laser Flow Cytophotometry,” Immunoassays in the Clinical Laboratory, 185-189 (Liss 1979); Wilson et al., “A New Microsphere-Based Immunofluorescence Assay Using Flow Cytometry,” J. Immunol. Meth. 107: 225-230 (1988); Fulwyler et al., “Flow Microsphere Immunoassay for the Quantitative and Simultaneous Detection of Multiple Soluble Analytes,” Meth. Cell Biol. 33: 613-629 (1990); Coulter Electronics Inc., United Kingdom Patent No. 1,561,042 (published Feb. 13, 1980); and Steinkamp et al., Review of Scientific Instruments 44(9): 1301-1310 (1973).

Similarly, methods of and instrumentation for applying and removing a magnetic field as part of an assay are known to those skilled in the art and reported in the literature. Examples of literature reports are Forrest et al., U.S. Pat. No. 4,141,687 (Technicon Instruments Corporation, Feb. 27, 1979); Ithakissios, U.S. Pat. No. 4,115,534 (Minnesota Mining and Manufacturing Company, Sep. 19, 1978); Vlieger, A. M., et al., Analytical Biochemistry 205:1-7 (1992); Dudley, Journal of Clinical Immunoassay 14:77-82 (1991); and Smart, Journal of Clinical Immunoassay 15:246-251 (1992). All of the citations in this and the preceding paragraph are incorporated herein by reference.

### VI. Solid Supports

Any type of solid support can be used in the invention. In some embodiments, the solid support is suitable for use in an ELISA assay. In some embodiments, the solid support is spherical or near-spherical. In some embodiments, the particles used in the practice of this invention are microscopic in size and formed of a polymeric material. Polymers that will be useful as microparticles are those that are chemically inert relative to the components of the biological sample and to the assay reagents other than the binding member coatings that are affixed to the microparticle surface. Suitable microparticle materials will also have minimal autofluorescence, will be solid and insoluble in the sample and in any buffers, solvents, carriers, diluents, or suspending agents used in the assay, and will be capable of affixing to the appropriate coating material. Examples of suitable polymers are polystyrenes, polyesters, polyethers, polyolefins, polyalkylene oxides, polyamides, polyurethanes, polysaccharides, celluloses, and polyisoprenes. Crosslinking is useful in many polymers for imparting structural integrity and rigidity to the microparticle. The size range of the microparticles can vary. In some embodiments, the microparticles range in diameter from about 0.3 micrometers to about 100 micrometers, e.g., from about 0.5 micrometers to about 40 micrometers, e.g., from about 2 micrometers to about 10 micrometers.

To facilitate the particle recovery and washing steps of the assay, the particles preferably contain a magnetically responsive material, i.e., any material that responds to a magnetic field. Separation of the solid and liquid phases, either after incubation or after a washing step, is then achieved by imposing a magnetic field on the reaction vessel in which the suspension is incubated, causing the particles to adhere to the wall of the vessel and thereby permitting the liquid to be removed by decantation or aspiration. Magnetically responsive materials of interest in this invention include paramagnetic materials, ferromagnetic materials, ferrimagnetic materials, and metamagnetic materials. Examples, include, e.g., iron, nickel, and cobalt, as well as metal oxides such as Fe₃O₄, BaFe₁₂O₁₉, CoO, NiO, Mn₂O₃, Cr₂O₃, and CoMnP.

The magnetically responsive material can be dispersed throughout the polymer, applied as a coating on the polymer surface or as one of two or more coatings on the surface, or incorporated or affixed in any other manner that secures the material in to the particle. The quantity of magnetically responsive material in the particle is not critical and can vary over a wide range. The quantity can affect the density of the microparticle, however, and both the quantity and the particle size can affect the ease of maintaining the microparticle in suspension for purposes of achieving maximal contact between the liquid and solid phase and for facilitating flow cytometry. An excessive quantity of magnetically responsive material in the microparticles may produce autofluorescence at a level high enough to interfere with the assay results. Therefore, in some embodiments, the concentration of magnetically responsive material is low enough to minimize any autofluorescence emanating from the material. With these considerations in mind, the magnetically responsive material in a particle in accordance with this invention is, for example, from about 0.05% to about 75% by weight of the particle as a whole. In some embodiments, the weight percent range is from about 1% to about 50%, e.g., from about 2% to about 25%, e.g., from about 2% to about 8%.

Coating of the particle surface with the appropriate assay reagent can be achieved by electrostatic attraction, specific affinity interaction, hydrophobic interaction, or covalent bonding. The polymer can be derivatized with functional groups for covalent attachment of the assay reagents by conventional means, notably by the use of monomers that contain the functional groups, such monomers serving either as the sole monomer or as a co-monomer. Examples of suitable functional groups are amine groups (—NH₂), ammonium groups (—NH₃⁺ or —NR₃⁺), hydroxyl groups (—OH), carboxylic acid groups (—COOH), and isocyanate groups (—NCO). Useful monomers for introducing carboxylic acid groups into polyolefins, for example, are acrylic acid and methacrylic acid.

Linking groups can be used as a means of increasing the density of reactive groups on the particle surface and decreasing steric hindrance. This may increase the range and sensitivity of the assay. Linking groups can also be used as a means of adding specific types of reactive groups to the solid phase surface if needed to secure the particular coating materials of this invention.

The capture agents can be directly or indirectly linked to the solid support via a linking agent. The capture agent and solid support can be conjugated via a single linking agent or multiple linking agents. For example, the capture agent and solid support may be conjugated via a single multifunctional (e.g., bi-, tri-, or tetra-) linking agent or a pair of complementary linking agents. In some embodiments, the capture agent and solid support are conjugated via two, three, or more linking agents. Suitable linking agents include, e.g., functional groups, affinity agents, stabilizing groups, and combinations thereof.

In some embodiments, an affinity agent (e.g., agents that specifically binds to a ligand) is the linking agent. In these embodiments, for example, a first linking agent is bound to the capture agent and a second linking agent is bound to the solid support. Affinity agents include receptor-ligand pairs, antibody-antigen pairs and other binding partners such as streptavidin/avidin and biotin. In some embodiments, the first linking agent is biotin and the second linking agent is streptavidin or avidin. In some embodiments, the first linking agent is a hapten (e.g., fluorescein) and the second linking agent is an anti-hapten (e.g., anti-fluorescein) antibody.

Functional groups include monofunctional linkers comprising a reactive group as well as multifunctional crosslinkers comprising two or more reactive groups capable of forming a bond with two or more different functional targets (e.g., peptides, proteins, macromolecules, semiconductor nanocrystals, or substrate). In some embodiments, the multifunctional crosslinkers are heterobifunctional crosslinkers comprising two different reactive groups.

Suitable reactive groups include, e.g., thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties. Amine reactive groups include, e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, e.g., haloacetyl and alkyl halide derivates, maleimides, aziridines, acryloyl derivatives, arylating agents, and thiol-disulfides exchange reagents. Carboxylate reactive groups include, e.g., diazoalkanes and diazoacetyl compounds, such as carbonyldiimidazoles and carbodiimides. Hydroxyl reactive groups include, e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, e.g., hydrazine derivatives for Schiff base formation or reduction amination. Active hydrogen reactive groups include, e.g., diazonium derivatives for Mannich condensation and iodination reactions. Photoreactive groups include, e.g., aryl azides and halogenated aryl azides, benzophenones, diazo compounds, and diazirine derivatives.

Other suitable reactive groups and classes of reactions useful in practicing the present invention are generally those that are well known in the art of bioconjugate chemistry. Currently favored classes of reactions available with reactive chelates are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

In some embodiments, the functional group is a heterobifunctional crosslinker comprising two different reactive groups that contain heterocyclic rings that can interact with peptides and proteins. For example, heterobifunctional crosslinkers such as N—[γ-maleimidobutyryloxy]succinimide ester (GMBS) or succinimidyl 4-[N-maleimidomethyl]cyclohexane-1-carboxylate (SMCC) comprise an amine reactive group and a thiol-reactive group that can interact with amino and thiol groups within peptides or proteins. Additional combinations of reactive groups suitable for heterobifunctional crosslinkers include, for example, carbonyl and sulfhydryl reactive groups; amine and photoreactive groups; sulfhydryl and photoreactive groups; carbonyl and photoreactive groups; carboxylate and photoreactive groups; and arginine and photoreactive groups. Examples of suitable useful linking groups are polylysine, polyaspartic acid, polyglutamic acid and polyarginine. N-hydroxysuccinimide (NHS), CMC 1-cyclohexyl-3-(2-morpholinoethyl)carbodiimide (CMC), N-Hydroxybenzotriazole (HOBt), and/or other crosslinking agents may be used.

In some embodiments, care is taken to avoid the use of particles that exhibit high autofluorescence. Particles formed by conventional emulsion polymerization techniques from a wide variety of starting monomers are generally suitable since they exhibit at most a low level of autofluorescence. Conversely, particles that have been modified to increase their porosity and hence their surface area, i.e., those particles that are referred to in the literature as “macroporous” particles, are less desirable since they tend to exhibit high autofluorescence. A further consideration is that autofluorescence increases with increasing size and increasing percentage of divinylbenzene monomer.

Multiplexing with the use of microparticles in accordance with this invention can be achieved by designing each particle (i.e., the “first” particle, and the “second” particle, and if relevant, the “third” particle, and the “fourth” particle, etc.) to have a distinctive differentiation parameter, which renders that group distinguishable from the other groups by flow cytometry.

One example of a differentiation parameter is the particle diameter, the various particle groups being defined by nonoverlapping diameter subranges. The widths of the diameter subranges and the spacing between mean diameters of adjacent subranges are selected to permit differentiation of the subranges by flow cytometry, and will be readily apparent to those skilled in the use of and instrumentation for flow cytometry. In this specification, the term “mean diameter” refers to a number average diameter. In some embodiments, the subrange width is about ±5% CV or less of the mean diameter, where “CV” stands for “coefficient of variation” and is defined as the standard deviation of the particle diameter divided by the mean particle diameter, times 100 percent. The minimum spacing between mean diameters among the various subranges can vary depending on the microparticle size distribution, the ease of segregating microparticles by size for purposes of attaching different assay reagents, and the type and sensitivity of the flow cytometry equipment. In some embodiments, best results will be achieved when the mean diameters of different subranges are spaced apart by at least about 6% of the mean diameter of one of the subranges, e.g., at least about 8% of the mean diameter of one of the subranges, e.g., at least about 10% of the mean diameter of one of the subranges. In some embodiments, the standard deviation of the particle diameters within each subrange is less than one third of the separation of the mean diameters of adjacent subranges.

Another example of a differentiation parameter that can be used to distinguish among the various groups of particles is fluorescence. Differentiation is accomplished by incorporating one or more fluorescent materials in the particles, the fluorescent materials having different fluorescent emission spectra and being distinguishable on this basis.

Fluorescence can in fact be used both as a means of distinguishing the particle groups from each other and as a means of detection and quantification for the assay performed on the particles. The use of fluorescent materials with different emission spectra can serve as a means of distinguishing the particle groups from each other and also as a means of distinguishing the particle group's classification from the (e.g., fluorescent) assay reported signals. An example of a fluorescent substance that can be used as a means of distinguishing particle groups is fluorescein and an example of a substance that can be used for the assay detection is phycoerythrin. In the use of this example, different particle groups can be dyed with differing concentrations of fluorescein to distinguish them from each other, while phycoerythrin is used as the label on the various labeled binding members used in the assay.

Still other examples of a differentiation parameter that can be used to distinguish among the various groups of particles are light scatter, or a combination of light scatter. Side angle light scatter varies with particle size, granularity, absorbance and surface roughness, while forward angle light scatter is mainly affected by size and refractive index. Thus, varying any of these qualities can serve as a means of distinguishing the various groups. Light emission can be varied by incorporating fluorescent materials in the microparticles and using fluorescent materials that have different fluorescence intensities or that emit fluorescence at different wavelengths, or by varying the amount of fluorescent material incorporated. By using fluorescence emissions at different wavelengths, the wavelength difference can be used to distinguish the particle groups from each other, while also distinguishing the labels in the labeled binding members from the labels that differentiate one particle group from another.

In a variation of the above, the microparticles will have two or more fluorochromes incorporated within them so that each microparticle in the array will have at least three differentiation parameters associated with it, i.e., light scatter together with fluorescent emissions at two separate wavelengths. For example, the microparticle can be made to contain a red fluorochrome such as Cy5 together with a far-red fluorochrome such as Cy5.5. Additional fluorochromes can be used to further expand the system. Each microparticle can thus contain a plurality of fluorescent dyes at varying wavelengths.

Still another example of a differentiation parameter that can be used to distinguish among the various groups of particles is absorbance. When light is applied to microparticles the absorbance of the light by the particles is indicated mostly by the strength of the laterally (side-angle) scattered light while the strength of the forward-scattered light is relatively unaffected. Consequently, the difference in absorbance between various colored dyes associated with the microparticles is determined by observing differences in the strength of the laterally scattered light.

A still further example of a differentiation parameter that can be used to distinguish among the various groups of particles is the number of particles in each group. The number of particles of each group is varied in a known way, and the count of particles having various assay responses is determined. The various responses are associated with a particular assay by the number of particles having each response.

As the above examples illustrate, a wide array of parameters or characteristics can be used as differentiation parameters to distinguish the microparticles of one group from those of another. The differentiation parameters may arise from particle size, from particle composition, from particle physical characteristics that affect light scattering, from excitable fluorescent dyes or colored dyes that impart different emission spectra and/or scattering characteristics to the microparticles, or from different concentrations of one or more fluorescent dyes. When the distinguishable microparticle parameter is a fluorescent dye or color, it can be coated on the surface of the microparticle, embedded in the microparticle, or bound to the molecules of the microparticle material. Thus, fluorescent microparticles can be manufactured by combining the polymer material with the fluorescent dye, or by impregnating the microparticle with the dye. Microparticles with dyes already incorporated and thereby suitable for use in the present invention are commercially available, from suppliers such as Spherotech, Inc. (Libertyville, Ill., USA) and Molecular Probes, Inc. (Eugene, Oreg., USA).

### VII. Reaction Mixtures

The present invention also provides for reaction mixtures used in the assays of the invention. Such mixtures comprise one or more of the components of the above-described method in the same aqueous reaction mixture, optionally in a mixture with a biological sample or a component thereof. In some embodiments, the reaction mixture comprises a biological sample from a human, and an anti-CA-125 capture agent (including but not limited to an antibody) and, optionally in the same or parallel reaction mixture, additional biomarker proteins (e.g., anti-IGFBP-2 or anti-prolactin or anti-osteopontin) capture agent (including but not limited to an antibody). In some embodiments, the two capture agents are linked to the same or different solid supports. In some embodiments, the solid support(s) is a bead. In some embodiments, the reaction mixture comprises the above-described capture agents, binding CA-125 and the confirmatory marker protein from a biological sample, further comprising detection agents for each of CA-125 and confirmatory marker protein as described elsewhere herein. In some embodiments, the capture agents are detectably labeled.

In some embodiments, a reaction mixture of the invention comprises a biological sample from a human and one or more antigens that are specifically recognized by an autoantibody that is expressed in ovarian cancer patients. In some embodiments, the reaction mixture further comprises one or more immunogenic fragments from the antigen(s). In some embodiments, the antigens are SBP1, p53, and IGF2BP2. In some embodiments, the antigens are linked to a solid support, e.g., a bead. In some embodiments, the antigens are linked to the same solid support. Antigens can be selected from those described elsewhere herein or can include other antigens recognized by an autoantibody that is expressed in ovarian cancer patients. The reaction mixture can further include at least one autoantibody binding to one of the antigens on the solid support, as well as a detection agent binding the autoantibody, optionally labeled or otherwise including a labeling reagent. The detection agent can be an antibody that specifically recognizes the antigen or can be an anti-human IgG antibody.

Other possible components of the reaction mixture will be clear from the remainder of this document.

### VIII. Kits

The present invention also provides for kits of performing the methods of the invention as described herein and can include any combination of the reagents described herein.

In some embodiments, the kit comprises an anti-CA-125 capture agent (including but not limited to an antibody) and/or an anti-confirmatory marker protein capture agent (e.g., anti-IGFBP-2 or anti-prolactin or anti-osteopontin). In some embodiments, the capture agent is an antibody. In some embodiments, the two capture agents will be linked to the same or different solid supports. In some embodiments, the solid support(s) is a bead. The kit can also include relevant detection agents for each of CA-125 or additional biomarker protein as described elsewhere herein. In some embodiments, the capture agents are detectably labeled.

In some embodiments, the kits further include one or more antigens that are specifically recognized by an autoantibody that is expressed in ovarian cancer patients. In some embodiments, the kit comprises one or more antigen, and/or a polypeptide comprising an immunogenic peptide thereof, selected from SBP1, p53, and IGF2BP2. In some embodiments, the antigens are linked to a solid support, e.g., a bead. In some embodiments, the kit comprises 2, 3, 4, 5, 6, 7, 8, 9, 10, 11, 12, 13, 14, 15, 16 or more antigens. Antigens can be selected from those described elsewhere herein or can include other antigens recognized by an autoantibody that is expressed in ovarian cancer patients. The kits can further include a detection agent, optionally labeled or otherwise including a labeling reagent as well). The detection agent can be an antibody that specifically recognizes the antigen or can be an anti-human IgG antibody.

Other possible components of the kit will be clear from the remainder of this document.

### IX. Computer-Based Methods

The calculations for the diagnostic methods described herein can involve computer-based calculations and tools. For example, once the levels of CA-125 and a confirmatory marker(s) and/or autoantibodies are detected, the levels can be compared by a computer to a threshold value, for example as described herein (for example a specific value determined based on percentile as found in healthy individuals). The tools can be advantageously provided in the form of computer programs that are executable by a general purpose computer system (referred to herein as a “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### EXAMPLES

The following examples are offered to illustrate, but not to limit the claimed invention.

### Example 1

### IGFBP-2 is Useful as a Confirmatory Marker for CA-125

At least two references have concluded that IGFBP-2 is not a useful tumor marker. See, Matuschek, C., et al., Eur. J. Med. Res., 16:451-456 (2011); Tworoger, S., et al., Cancer Epidemiol. Biomarkers Prev. 16:1691-1695 (2007). However, we determined that IGFBP-2 is useful, in combination with CA-125, for detection of cancer.

Fifty microliters of buffer and serum, diluted 1:10 (for CA-125) or 1:400 (for IGFBP-2) with buffer, was incubated with a dyed bead mixture consisting of beads separately coated with anti-CA-125 or anti-IGFBP2. After two hours at room temperature, the beads were washed and then incubated 30 minutes with secondary antibodies to these three proteins, labeled with biotin (for CA-125 and IGFBP-2). After a wash step, the beads were treated with SA-PE to produce fluorescent signal. Prolactin and osteopontin (discussed in Table 4) were detected using the same protocol as used to detect IGFBP-2.

Separately, 50 μL of serum, diluted 1:60 with buffer, was incubated with a dyed bead mixture consisting of beads separately coated with either SBP-1 or p53. After 1 hour at room temperature, the beads were washed and then incubated 30 minutes with anti-human IgG bound to PE, in order to produce fluorescent signal.

Table 1 provides a summary of the results:

TABLE 1

| | | | | | |
|---|---|---|---|---|---|
| Sample | Cancer stage | CA-125 (IU/mL) | anti-SBP-1 RFI | anti-p53 RFI | IGFBP-2 (ng/mL) |

Normal

| | | | | | |
|---|---|---|---|---|---|
| Ov_N_045 | healthy female | 2 | 66 | 82 | 451 |
| Ov_N_046 | healthy female | 11 | 94 | 72 | 529 |
| Ov_N_047 | healthy female | 9 | 58 | 85 | 973 |
| Ov_N_048 | healthy female | 9 | 43 | 74 | 651 |
| Ov_N_049 | healthy female | 8 | 65 | 123 | 803 |
| Ov_N_051 | healthy female | 6 | 82 | 477 | 339 |
| Ov_N_055 | healthy female | 4 | 101 | 149 | 446 |
| Ov_N_057 | healthy female | 5 | 45 | 77 | 1131 |
| Ov_N_058 | healthy female | 8 | 86 | 156 | 479 |
| Ov_N_059 | healthy female | 6 | 78 | 1312 | 212 |
| Ov_N_060 | healthy female | 8 | 46 | 73 | 376 |
| Ov_N_061 | healthy female | 3 | 94 | 519 | 933 |
| Ov_N_063 | healthy female | 4 | 36 | 134 | 1194 |
| Ov_N_065 | healthy female | 5 | 68 | 93 | 534 |
| Ov_N_066 | healthy female | 5 | 63 | 95 | 606 |
| Ov_N_068 | healthy female | 10 | 44 | 319 | 1133 |
| Ov_N_069 | healthy female | 10 | 50 | 99 | 888 |
| Ov_N_070 | healthy female | 9 | 60 | 74 | 1009 |
| Ov_N_071 | healthy female | 9 | 56 | 5212 | 657 |
| Ov_N_072 | healthy female | 9 | 40 | 1957 | 816 |
| Ov_N_073 | healthy female | 6 | 95 | 1082 | 450 |
| Ov_N_075 | healthy female | 10 | 53 | 56 | 869 |
| Ov_N_076 | healthy female | 6 | 62 | 197 | 847 |
| Ov_N_077 | healthy female | 5 | 67 | 77 | 281 |

OVCA

| | | | | | |
|---|---|---|---|---|---|
| Ov00349 | 2 | 8 | 324 | 152 | 1045 |
| Ov00110 | 4 | 8 | 57 | 118 | 937 |
| Ov00354 | 3 | 9 | 62 | 929 | 562 |
| Ov00351 | 2 | 9 | 47 | 84 | 1206 |
| Ov00347 | 2 | 10 | 63 | 77 | 768 |
| Ov00112 | 1 | 10 | 1909 | 80 | 1060 |
| Ov00196 | 3 | 15 | 415 | 210 | 1058 |
| Ov00324 | 2 | 30 | 2411 | 106 | 865 |
| Ov00329 | 1 | 32 | 278 | 166 | 278 |
| Ov00069 | 1 | 34 | 208 | 117 | 328 |
| Ov00325 | 2 | 37 | 28 | 85 | 1026 |
| Ov00331 | 2 | 39 | 40 | 56 | 1271 |
| Ov00326 | 2 | 44 | 66 | 136 | 689 |
| Ov00345 | 2 | 49 | 34 | 99 | 714 |
| Ov00084 | 4 | 52 | 106 | 144 | 1321 |
| Ov00348 | 2 | 59 | 103 | 267 | 841 |
| Ov00350 | 1 | 60 | 48 | 80 | 1310 |
| Ov00363 | 3 | 61 | 47 | 92 | 1412 |
| Ov00372 | 3 | 62 | 174 | 243 | 1162 |
| Ov00368 | 1 | 65 | 416 | 129 | 1693 |
| Ov00367 | 3 | 74 | 82 | 175 | 2963 |
| Ov00149 | 3 | 74 | 65 | 156 | 1891 |
| Ov00334 | 3 | 86 | 57 | 27110 | 905 |
| Ov00432 | 1 | 87 | 39 | 62 | 246 |
| Ov00346 | 2 | 88 | 50 | 133 | 882 |
| Ov00186 | 3 | 92 | 107 | 3451 | 3221 |
| Ov00430 | 1 | 92 | 74 | 107 | 647 |
| Ov00134 | 3 | 97 | 64 | 89 | 5434 |
| Ov00306 | 3 | 99 | 71 | 103 | 2871 |
| Ov00343 | 1 | 101 | 71 | 7 | 35 |
| Ov00342 | 2 | 157 | 33 | 59 | 1145 |
| Ov00425 | 4 | 195 | 23 | 56 | 2560 |
| Ov00332 | 3 | 207 | 41 | 115 | 1505 |
| Ov00418 | 2 | 285 | 374 | 145 | 1360 |
| Ov00344 | 3 | 286 | 43 | 68 | 549 |
| Ov00365 | 4 | 287 | 81 | 28776 | 901 |
| Ov00421 | 2 | 322 | 61 | 77 | 4363 |
| Ov00435 | 2 | 390 | | | 1365 |
| Ov00330 | 2 | 409 | 1129 | 462 | 1426 |
| Ov00323 | 2 | 599 | 56 | 67 | 1916 |
| Ov00426 | 4 | 1125 | 31 | 62 | 1078 |
| Ov00375 | 3 | 1643 | 9441 | 278 | 1963 |
| Ov00320 | 3 | 1704 | 122 | 26913 | 1557 |
| Ov00305 | 4 | 2406 | 49 | 130 | 2709 |
| Ov00310 | 4 | 2806 | 32 | 174 | 4870 |
| Ov00420 | 2 | 4771 | 135 | 2705 | 3672 |
| Ov00356 | 3 | 5118 | 55 | 28212 | 1526 |
| Ov00361 | 3 | 9048 | 47 | 112 | 1370 |
| | cutoff: | 1) 35 (98th pctile) | 200 | 10000 | 1200 |
| | | 2) 100 (99.9th pctile) | | | |

For CA-125, we identified 19 ovarian cancer samples with a result of 100 IU/mL or higher. As discussed previously, these samples exceed the 99.9% percentile in a healthy population and therefore are treated as positive. Then we identified samples that had an CA-125 result higher than 30 and less than 100 IU/mL. These results were treated as positive only if the IGFBP2 level exceed the 99.9^{th} percentile in our own reference range (patients labeled “normal”). The 99.9^{th} percentile was identified in this study as 1200 ng/mL (see bottom of table). Out of 19 patients with CA-125 results in this range we identified 10/19 with elevation of this confirmatory marker, and these were treated as positive.

Finally, for samples with a CA-125 result below 100, we established an autoantibody 99^{th} percentile for anti-SBP-1 and for anti-p53. These cutoffs are shown at the bottom of the table (because of the small number of normals, the values shown are actually slightly above the 98^{th} percentile). The data indicate that 6/10 samples with CA-125 results below 35 IU/mL and 1/19 samples with CA-125 results between 35 and 100 had an elevated level of anti-SBP-1. Similarly, in this set, one sample with a CA-125 of 86 had elevated anti-p53.

For the sera shown, CA-125, which is known to have no better than 98% specificity, was positive for 38/48 samples (79% sensitivity). The method proposed here was positive for 37/48 samples (77% sensitivity) but has the potential to have specificity >99.5% because of the use of internal confirmation.

In summary, internal confirmation of results to obtain good sensitivity was achieved while obtaining high specificity.

### Example 2

### Autoantibodies Improve Sensitivity of CA-125-Based Cancer Detection

Selenium Binding Protein 1 (SBP1) was used to screen for autoantibodies in ovarian cancer and healthy patients. SBP1 autoantibodies (AAbs) have been shown previously in patients having infertility and premature ovarian failure (Edassery, S., et al., Fertil. Steril. 94(7):2636-2641 (2010)) and also with ovarian cancer (Barua, A., et al., Am. J. Reproduct. Immunol. 57:243-249 (2007)). We have determined that many patients with such AAbs do not have CA-125 elevation; therefore, detection of SBP1 autoantibodies substantially increase sensitivity for ovarian cancer detection. Data for SBP1 autoantibodies is shown in Table 1.

In addition, we have evaluated another protein (p53) for which AAbs have been previously described in ovarian cancer. See Table 1. We have found that when level of CA-125 below 30 or 35 IU/mL were considered, there are a number of patients where p53 AAbs adds sensitivity.

### Example 3

### Autoantibodies' Specificity can be Improved by Separately Detecting Autoantibodies with an Antigen and an Immunogenic Peptide of the Antigen

Using AAbs for ovarian cancer detection poses the same risk of false positive results as CA-125 measurement because there will be patients with results above the 98th percentile for CA-125 who are healthy, or have a different type of cancer, or other clinical condition. We propose an approach to improve the specificity of these biomarkers. This approach is based on the observation that an AAb against a protein can be confirmed by demonstrating the presence of an AAb against a specific peptide, known to be an immunoepitope, for that protein. We have demonstrated that using the known immunoepitope for p53 allowed a substantial reduction in false positive results obtained when the protein was used alone (see Table 2). The patient cohort consisted of 937 apparently healthy women, 420 women with benign masses, and 507 women with ovarian cancer. Using autoantibodies to p53, as well as its immunoepitope (SEQ ID NO:1), the false positive rate was 1/937 or 0.11% for healthy women and 2/420 or 0.47% for women with benign masses (1 of these was also positive for CA-125 with a result over 100 IU/mL). The pair (full length and immunoepitope) detected 6 samples that had CA-125 results below 35 IU/mL and would have been missed using full-length alone, along with 3 samples that had CA-125 results between 35 and 100 IU/mL.

TABLE 2
Detection of p53 Antibodies (protein, immunoepitope)
in conjunction with CA-125

| | | | CA125 | | |
|---|---|---|---|---|---|
| | | | (Co-Dev) | p53 | p53 |
| Sample | Age | Stage | IU/mL | (protein) | peptide |
| 17836 | 59 | Healthy | 6 | 3697 | 1899 |
| | | Female | | | |
| Ov00194 | 42 | Benign | 441 | 25997 | 26911 |
| V3514 | 49 | Benign | 13 | 11281 | 11268 |
| Ov00302 | 55 | IC | 4 | 23633 | 6054 |
| Ov_GOG_235 | 71 | IA | 4 | 23845 | 10535 |
| Ov00088 | 51 | IIA | 12 | 2773 | 1103 |
| Ov_GOG_358 | 76 | IVB | 17 | 10406 | 6178 |
| Ov00230 | 68 | I | 23 | 8617 | 3139 |
| V4867 | 51 | IIC | 32 | 9716 | 17258 |
| V2814 | 44 | IIIC | 61 | 24486 | 23097 |
| Ov00334 | 50 | III | 86 | 23919 | 22343 |
| V3853 | 47 | IIB | 98 | 25422 | 26047 |
| Ov_GOG_263 | 76 | IA | 109 | 2733 | 3468 |
| Ov00146 | 52 | IIIC | 147 | 25316 | 16210 |
| Ov_GOG_278 | 39 | IIB | 189 | 24181 | 24638 |
| Ov_GOG_200 | 59 | IIB | 193 | 21928 | 6063 |
| Ov00051 | 50 | III | 251 | 24154 | 23957 |
| Ov_GOG_370 | 58 | IIIC | 252 | 24914 | 26173 |
| Ov00377 | 65 | IIA | 317 | 26210 | 28471 |
| Ov_GOG_378 | 73 | IIIC | 402 | 4546 | 3240 |
| Ov_GOG_325 | 54 | IIIC | 409 | 17063 | 6974 |
| Ov_GOG_314 | 66 | IIIC | 422 | 13191 | 22198 |
| Ov00068 | 50 | IV | 439 | 23977 | 23507 |
| V5087 | 74 | IIIC | 440 | 11825 | 3658 |
| Ov_GOG_330 | 81 | IIIC | 461 | 23716 | 7670 |
| Ov00108 | 60 | III | 488 | 23240 | 24642 |
| V3166 | 79 | IIIC | 530 | 24398 | 1393 |
| Ov_GOG_311 | 45 | IIIA | 701 | 26217 | 27403 |
| Ov00261 | 55 | II | 765 | 24280 | 21363 |
| Ov_GOG_371 | 63 | IIIC | 859 | 25950 | 27345 |
| V2329 | 61 | IV | 925 | 24714 | 26457 |
| Ov_GOG_348 | 74 | IVA | 968 | 26202 | 27077 |
| Ov00055 | 71 | IV | 1062 | 15677 | 8044 |
| J6958 | 60 | IV | 1242 | 27599 | 28061 |
| Ov00303 | 66 | IIIC | 1413 | 5082 | 1695 |
| Ov_GOG_321 | 59 | IIIC | 1431 | 23934 | 26657 |
| Ov00320 | 59 | III | 1704 | 24185 | 26892 |
| Ov_GOG_350 | 67 | IIIC | 1912 | 18495 | 3521 |
| Ov00395 | 55 | I | 2060 | 23950 | 17746 |
| Ov_GOG_322 | 48 | IIIC | 2451 | 14827 | 15415 |
| Ov_GOG_381 | 52 | IIIC | 2704 | 10292 | 1422 |
| Ov_GOG_382 | 59 | IIIC | 3872 | 23795 | 14034 |
| Ov00356 | 69 | IIIC | 5118 | 24592 | 24851 |
| Ov_GOG_197 | 48 | IIB | 5454 | 25522 | 28011 |
| Ov00386 | 75 | II | 5703 | 26849 | 27514 |

1) 35 (98th pctile) 1554 1026 Cutoffs

### Example 4

We have conducted a large study of sera from apparently healthy women (⅔ age 55 or older) and women with established ovarian cancer. In this study we considered results for the proteins CA-125 and IGFBP2 (measured as described above) as well as two additional proteins osteopontin and prolactin that have been described in other studies. We measured autoantibodies against SBP-1 as described above, against p53 as described above, and against two peptides found in p53 that have immunogenic epitopes. In addition, we measured antibodies against an immunogenic epitope of the protein IGF2BP2, which have not been previously described. For CA-125, we considered any sample with a CA-125 result over 100 IU/mL as positive for ovarian cancer.

For samples with a CA-125 result from 30 to 100 IU/mL, we considered samples positive for ovarian cancer if they:

(1) had a positive protein level (IGFBP2, prolactin, or osteopontin); or

(2) were positive for an autoantibody (i.e., autoantibodies that bound p53, SBP1 or IGF2BP2 and bound a corresponding immunogenic peptide thereof.

For samples with a CA-125 result below 30 IU/mL, we considered samples positive for ovarian cancer if the sample:

(1) was positive autoantibodies for p53 and one of p53's epitopes; or

(2) was positive for a confirmatory protein (IGFBP2, prolactin, or osteopontin) and positive for an SBP1 or IGF2BP2 autoantibody. For all of the non-CA-125 analytes, cutoffs were established corresponding to the 99^{th} percentile or 99.8^{th} percentile of healthy sera.

In addition to testing healthy individuals we tested sera from patients with conditions that can produce elevated levels of CA-125 in the absence of ovarian cancer. These patients had been diagnosed with SLE, RA, or PID.

The results of this study are shown below in Table 3. The results of CA-125 measurement only (using the commonly accepted cutoff of 35 IU/mL) are compared to results obtained using the algorithm described in section 125 above in Table 4.

TABLE 3
EVALUATION OF SERA FROM HEALTHY WOMEN,
WOMEN WITH OVARIAN CANCER AND OTHER CLINICAL
CONDITIONS THAT MAY PRODUCE AN ELEVATED CA-125

| | | | | patients | | |
|---|---|---|---|---|---|---|
| | | | | positive | patients | |
| | | | patients | for p53 | positive | |
| | | | positive | (protein | for any | patients |
| | | CA | for any | and 1 + | non-p53 | Positive by |
| | N | 125 >35 | Protein | peptide)` | Aab | algorithm |
| Healthy | 359 | 0 | 6 | 1 | 5 | 1 |
| women | | | | | | |
| CA 125 | 47 | 0 | 4 | 8 | 0 | 8 |
| <30 | | | | | | |
| CA 125 30 | 38 | 32 | 19 | 2 | 6 | 24 |
| to 100 | | | | | | |
| CA 125 | 212 | 212 | 115 | 19 | 11 | 212 |
| >100 | | | | | | |
| PID | 20 | 8 | 2 | 0 | 0 | 5 |
| RA | 20 | 2 | 4 | 0 | 0 | 3 |
| SLE | 20 | 3 | 2 | 0 | 0 | 1 |

In Table 3, “patients positive for any protein” refers to the presence of IGF2BP2, prolactin or osteopontin in the patient sample at a level above the 99^{th} or 99.8^{th} percentile of healthy patient sera.

TABLE 4
SUMMARY OF SERA FROM HEALTHY WOMEN, WOMEN
WITH OVARIAN CANCER AND OTHER CLINICAL CONDITIONS
THAT MAY PRODUCE AN ELEVATED CA-125

| | | | Patients Positive by |
|---|---|---|---|
| | N | CA 125 >35 | algorithm |
| Healthy women | 359 | 0 | 1 |
| CA 125 <30 | 47 | 0 | 8 |
| CA 125 30 to 100 | 38 | 32 | 24 |
| CA 125 >100 | 212 | 212 | 212 |
| PID | 20 | 8 | 5 |
| RA | 20 | 2 | 3 |
| SLE | 20 | 3 | 1 |
| Total non-cancer | 419 | 13 | 9 |
| total cancer | 297 | 244 | 244 |

The data in table 3 shows that the proteins and autoantibodies described herein are occasionally detected in healthy women, but p53 protein/peptide are rarely detected in healthy women and there were no samples in which both proteins and autoantibodies were detected. In contrast, both the proteins and the autoantibodies were commonly observed in patients with cancer.

Using the algorithm described above, the number of cancer patients detected by the algorithm (244 out of 295) was identical to the number detected using the traditional method of CA-125 measurement with a cutoff of 35 IU/mL.

Further, when samples with elevated CA-125 in the absence of cancer were considered, the algorithm was able eliminate 6 out of 13 samples with a CA-125 level between 30 and 100 IU/mL because they were not positive by the algorithm (no positive proteins or autoantibodies). Only samples that had a CA-125 greater than 100 IU/mL were algorithm positive in this group.

In summary, this large data set demonstrates that the methods described here offer sensitivity equivalent to the conventional approach but offer superior specificity for samples that may have elevated CA-125 in the absence of cancer.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A method of detecting the presence or absence of ovarian cancer in an individual human, the method comprising,
detecting the level of the following agents in a biological sample from the individual:
a. CA-125; and
b. IGFBP-2 or prolactin or osteopontin; and
correlating the level of the agents to the presence, absence, or stage of ovarian cancer in the individual wherein the correlating comprises using the IGFBP-2 or prolactin or osteopontin levels as a confirmatory criterion for higher than normal levels of CA-125.

2. The method of claim 1, further detecting in a biological sample from the individual the level of:
at least one autoantibody specific for a target antigen protein, wherein an elevated level of the autoantibody specific for the target antigen protein is indicative of cancer.

3. The method of claim 1, wherein the correlating comprises determining whether the level of CA-125 is below about 30 IU/mL serum, between about 30 and 100 IU/mL serum, or over about 100 IU/mL serum, and whether IGFBP-2 or prolactin or osteopontin levels are above normal levels, wherein the presence of ovarian cancer is indicated by:
a CA-125 level over about 100 IU/mL; or
a CA-125 level between about 30 and 100 IU/mL and an IGFBP-2 or prolactin or osteopontin level are above the normal level.

4. The method of claim 2, wherein autoantibodies specific for the target antigen protein are separately captured by:
the target antigen protein; and
the immunogenic fragment thereof; and
the detecting comprises separately detecting binding of the autoantibodies to the target antigen protein and to the immunogenic fragment thereof.

5. The method of claim 2, wherein more than one target antigen protein for more than one different autoantibodies are linked to the solid support, thereby detecting the level of more than one autoantibody in the sample.

6. The method of claim 2, wherein the autoantibody target antigen protein is SBP1, p53, or IGF2BP2.

7. The method of claim 5, wherein the immunogenic fragment comprises SEQ ID NO:1, 2, or 3.

8. A kit for detecting cancer in a human individual, the kit comprising,
anti-CA-125 antibody; and
an anti-IGFBP-2 or anti-prolactin or osteopontin antibody.

9. The kit of claim 8, further comprising:
a target antigen protein, or an immunogenic fragment thereof, that specifically detects an autoantibody that occurs at a higher rate in individuals having cancer compared to individuals not having cancer.

10. The kit of claim 9, comprising the antigen and the immunogenic fragment thereof.

11. The kit of claim 9, wherein the immunogenic fragment comprises SEQ ID NO:1, 2, or 3.

12. The kit of claim 9, wherein the kit further comprises two or more different antigens and/or immunogenic fragments thereof, each of which specifically detect a different autoantibody that occurs at a higher rate in individuals having cancer compared to individuals not having cancer.

13. The kit of claim 12, wherein the two or more different antigens, or immunogenic fragments thereof, are linked to the same solid support.

14. The kit of claim 9, wherein the antigen is SBP1, p53, or IGF2BP-2.

15. The kit of claim 8, wherein the anti-CA-125 antibody and the anti-IGFBP-2 or anti-prolactin or osteopontin antibody are linked to the same solid support.

16. The kit of claim 8, wherein the anti-CA-125 antibody and the IGFBP-2 or prolactin or osteopontin antibody are linked to different solid supports.

17. The kit of claim 16, wherein the solid support is a plurality of beads, the beads comprising a bead linked to the anti-CA-125 antibody and a bead linked to the IGFBP-2 antibody, wherein the bead linked to the anti-CA-125 antibody is distinguishable from the bead linked to the IGFBP-2 antibody by flow cytometry.

18. The kit of claim 8, further comprising an anti-human IgG antibody.

19. The kit of claim 8, wherein the anti-human IgG antibody is linked to a detectable label.

20. A method of detecting the presence or absence of ovarian cancer in an individual human, the method comprising,
detecting the level of the following agents in a biological sample from the individual:
a. CA-125; and
b. two or more of: autoantibodies specific for SBP1, p53 or IGF2BP2, correlating the level of the agents to the presence, absence, or stage of ovarian cancer in the individual.

21. The method of claim 20, wherein detecting the autoantibodies comprises contacting a sample to an antigen selected from SBP1, p53 or IGF2BP2, and contacting the sample to one or more immunogenic fragment of the antigen.
